(19) United States
(12) Patent Application Publication
(10) Pub. No.: US 2010/0226979 A1
(43) Pub. Date: Sep. 9, 2010

(54) Taste Masked Phamaceutical Composition for Oral Solid Dosage form and Process for Preparing the Same Using Magnesium Aluminium Silicate

(75) Inventors: Namdev Kashid, Maharashtra (IN); Pradeep Chouhan, Madhya Pradesh (IN); Gour Mukherji, Haryana (IN)

Correspondence Address: COHEN, PONTANI, LIEBERMAN & PAVANE LLP, 551 FIFTH AVENUE, SUITE 1210, NEW YORK, NY, 10176, US

(73) Assignee: JUBILANT ORGANOSYS LIMITED, Noida (UP) (IN)

(21) Appl. No.: 12/225,288

(22) PCT Filed: Mar. 19, 2007
(86) PCT No.: PCT/IN2007/000109

§ 371 Date: Sep. 17, 2008

(30) Foreign Application Priority Data
Mar. 21, 2006 (IN) 752/DEL/2006

(51) Int. Cl. A61K 9/20; A61K 31/519; A61P 25/18; B29B 9/16

(52) U.S. Cl. 424/465; 514/259.41; 264/6

(57) ABSTRACT

Disclosed herein a taste masked pharmaceutical composition suitable for oral solid dosage form comprising adsorbate of unpleasant or objectionable tasting active pharmaceutical agents and water insoluble polymer, wherein said active is first blended with an adsorbent such as magnesium aluminium silicate to achieve partially or significantly taste masking of said active and further granulated the resultant blend with water insoluble polymer to strengthen the taste masking without affecting the release of said active.

### FIELD OF THE INVENTION

This invention, in general, relates to a taste masked pharmaceutical compositions and the process for preparing the same. More particularly, the present invention provides a taste-masked composition suitable for an oral solid dosage form comprising objectionable tasting active pharmaceutical agent(s).

### BACKGROUND OF THE INVENTION

Orally administered therapeutic agents are given to a patient in numerous forms, in liquid forms such as liquid solutions, emulsions or suspensions or in solid forms such as capsules or compressed tablets. Medicaments administered in tablet or capsule form are frequently intended to be swallowed whole. Additionally, there are other solid forms, such as orally disintegrating tablets and chewable tablets, which are dissolved or crushed in the mouth and are not swallowed whole. Normally, for liquid products and the non-swallowing solid dosage forms, like the orally disintegrating or chewable tablets, the unpleasant or disagreeable or objectionable taste of the active pharmaceutical agent(s) need to be taken into account in formulating the medicine, especially the provision of means to prevent the taste from being apparent during the time that the medicine is in the mouth.

“Palatability” and “mouth feel” are extremely important factors for achieving total compliance for patients who are being administered the unpleasant or disagreeable or objectionable tasting active pharmaceutical agent(s). Patient compliance, which is essential for continuation and completion of any drug therapy, can be achieved by optimum taste masking of the unpleasant or disagreeable or objectionable tasting active pharmaceutical agent(s).

Therefore, it is a challenging task for the formulation scientist to provide highly palatable composition of the bitter or unpleasant or objectionable tasting active pharmaceutical ingredient without compromising the bioavailability of the bitter or unpleasant or objectionable tasting active pharmaceutical ingredient.

U.S. Pat. No. 3,558,600, assigned to Abbott laboratories describes a method for masking the bitter taste of antihistaminic agents belonging to the substituted 1-(p-chloro-benzhydryl)piperazine family, which consists in converting the active substance in free base form into the form of its salt with a long-chain alkyl sulfate, for example such as stearyl sulfate. This method has its own limitations, like poor solubility and reduced absorption of the drug. Besides, this approach cannot be successful for highly bitter drugs.

Well-known methods for taste masking, generally have involved coating of the particles of the active ingredient and/or the tablet containing such active ingredient, with various coating materials or combinations of coating materials.

In U.S. Pat. No. 4,656,027 (assigned to Astra Lakemedel Aktiebolag), there is provided a method and product wherein a basic substance is mixed with a bitter tasting drug, which is insoluble at high pH. The mixture is encapsulated with a polymer, which is a cellulose derivative, vinyl derivative or an acid soluble polymer such as a copolymer of dimethylaminoethyl methacrylate. In addition, a method for a mixture of polymer-coated drug with a basic substance is also claimed to give suspensions on reconstitution that give stable taste-masked products.

U.S. Pat. No. 4,693,896, assigned to FMC Corporation, teaches a method of taste masking by coating the ibuprofen tablets with aqueous dispersion of ethylcellulose (Aquacoat®). Similarly Japanese Patent No. 81/46,837 suggests coating of particles of ibuprofen with beta-cyclodextrin (i.e., beta-cyclodextrin) to provide for the taste masking of the drug.

U.S. Pat. No. 5,084,278, assigned to Nortec Development Associates, Inc., discloses the similar approach of coating the drug with polymers to mask their unpleasant taste.

U.S. Pat. No. 5,286,489, assigned to the Procter & Gamble Company describes a porous drug-polymer matrix formed by admixing one or more bitter tasting active ingredient and a methyl methacrylic ester copolymer in at least a 1:1 by weight ratio of active ingredient to copolymer, effective to mask the taste of the drug.

U.S. Pat. No. 6,767,557 assigned to Ortho-McNeil Pharmaceutical Inc, describes a taste masked pharmaceutical composition in the form of microcapsules, wherein the drug particles in the core of the microcapsules, are coated with taste masking effective amount of a weakly acidic methacrylic acid-ethyl acrylate copolymer.

In all the prior arts cited above coating the drug or dosage form with polymers usually restrict or delay the drug release from the dosage form thereby adversely affecting the bioavailability of the drug from the dosage form. Besides, the forces used to compress tablets can fracture the polymer coating, which reduces the effectiveness of the taste masking system.

U.S. Pat. No. 4,865,851, assigned to Glaxo Group Limited, teaches lipid-based micro encapsulation technique for taste masking of the bitter drugs wherein the drug (cefuroxime axetil) in particulate form is coated with an integral coating of lipid or a mixture of lipids. This technique requires highly sophisticated hot-melt granulation process for producing fine particles, and may have adverse effects on heat sensitive molecules or hinder drug release adversely.

Therefore, by the aforementioned facts there exists a necessity of a taste masked pharmaceutical compositions suitable for oral solid dosage form, which overcome the problem related to the prior arts. Accordingly, the present invention provides a taste masked pharmaceutical compositions suitable for oral solid dosage form, which obviates the drawback associated with prior arts.

### SUMMARY OF THE INVENTION

It is a principal aspect of the present invention to provide a taste masked pharmaceutical composition suitable for oral solid dosage form comprising one or more unpleasant or objectionable tasting active pharmaceutical agents, wherein said composition exhibit effective taste masking without affecting the release of said active.

Further aspect of the present invention is to provide a taste masked pharmaceutical composition suitable for oral solid dosage form comprising one or more unpleasant or objectionable tasting active pharmaceutical agents, wherein said composition comprising adsorbate of one or more unpleasant or objectionable tasting active pharmaceutical agents and water insoluble polymer, wherein said composition is characterized by providing desired taste masking the objectionable taste unaltering or unhindering the release rate of the said active.

In accordance with another aspect of the present invention, there is provided a taste masked pharmaceutical composition suitable for oral solid dosage form comprising one or more unpleasant or objectionable tasting active pharmaceutical agents, wherein drug adsorbate is prepared by adsorbing or of one or more objectionable tasting active pharmaceutical agents on an adsorbent.

In accordance with yet another aspect of the present invention, there is provided a taste masked pharmaceutical composition suitable for oral solid dosage form comprising one or more unpleasant tasting active pharmaceutical agents, wherein said active is first blended with said adsorbent to achieve partially or significantly taste masking of said active and further granulated the resultant blend with water insoluble polymer to strengthen the taste masking without affecting the release of said active.

In accordance with still another aspect of the present invention, there is provided a taste masked pharmaceutical composition suitable for oral solid, dosage form comprising one or more unpleasant tasting active pharmaceutical agents, wherein said composition is characterized by a homogeneous distribution of adsorbate of one or more active pharmaceutical active in the water insoluble polymer.

In accordance with yet another aspect of the present invention, there is provided a taste masked composition suitable for oral solid dosage form comprising one or more unpleasant or objectionable tasting active pharmaceutical agents, further comprising other pharmaceutically acceptable excipients or adjuvants for the extra granular composition in order to facilitate the formulation of various dosage forms of the active pharmaceutical agents like tablets, granules and capsules.

In accordance one preferred embodiment of the present invention, there is provided a process for the said taste masked composition suitable for oral solid dosage form, the process comprises of forming a adsorbate of one or more active with an adsorbent such as magnesium aluminum silicate, mixing the resultant adsorbate with lubricants, dispersant, surfactant, sweeteners, disintegrants and sifting the mixture through suitable size mesh, granulating the resultant mixture with solution of water insoluble polymer, drying the resultant granules and sifting them through suitable size mesh, further blending the formed granules with fillers, lubricants, antiadherants, disintegrants, sweeteners, alkalizers and flavoring agents and compressing the resultant blend into tablets of suitable shape or converting the blend into suitable oral solid dosage forms.

In accordance one preferred embodiment of the present invention, there is provided a process for the said taste masked composition suitable for oral solid dosage form, the process comprises of forming a adsorbate of one or more active with an adsorbent such as magnesium aluminum silicate, mixing the resultant adsorbate with dispersant, lubricants, sweeteners, disintegrants and passing the mixture through suitable size mesh, granulating the resultant mixture with aqueous solution of binder and surfactant, followed by further granulation with solution of water insoluble polymer, drying the resultant granules and sifting them through suitable size mesh, further blending the formed granules with fillers, lubricants, antiadherants, disintegrants, sweeteners, alkalizers and flavoring agents and compressing the resultant blend into tablets of suitable shape or converting the blend into suitable oral solid dosage forms.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

“Active pharmaceutical agents” in accordance with the present invention means any active pharmaceutical ingredients or their pharmaceutical acceptable salts, derivatives, solvates or hydrates, which elicit desired therapeutic response of a selected therapeutic category.

“Water insoluble polymer” in accordance with the present invention means polymer is practically insoluble in water at room temperature.

Unless otherwise stated all weight percentages as mentioned herein are based on the total weight of composition.

The present invention describes a unique taste masking composition suitable for oral solid dosage form comprising adsorbate of unobjectionable or unpleasant tasting active pharmaceutical agent and one or more water-insoluble polymers. Such a composition overcomes the problems associated with the prior art i.e., inadequate taste masking and restricted said active release from the dosage form, wherein said complex is prepared by adsorbing or blending partially or significantly of one or more objectionable tasting active pharmaceutical agents with an adsorbent such as magnesium aluminium silicate.

Applicants have found that taste masking composition suitable for oral solid dosage forms comprising for one or more bitter drugs without restricting drug release from the said dosage form by blending adsorbate of bitter drugs with water insoluble polymer. It has also been found by the inventors of present invention that for very bitter drugs, the amount of magnesium aluminum silicate required for taste masking will be very high. Such high amounts have very restricting influence in drug release from the dosage form, particulary, at pH conditions of 4.5 & above. Alternatively exclusive coating with water insoluble or water swellable polymer will necessarily require high amount of polymer application which will be either be very tedious or non feasible. Hence, incorporating both drug-magnesium aluminum silicate adsorbate and water insoluble polymer and optionally incorporating magnesium aluminum silicate or ion exchange resin or β-cyclodextrin in the extragranular composition, achieves the desired taste masking effect without the adverse properties that is (1) no adverse impact on drug release and (2) shorter processing time & convenient application of water insoluble polymer(s).

The adsorbate of active pharmaceutical agent can be formed by mixing or blending the active pharmaceutical agents with the magnesium aluminum silicate in a high or moderate shear mixers like planetary mixer or rapid mixer granulator (RMG). Alternatively, adsorbate can be formed by wet granulation involving magnesium silicate and active pharmaceutical agents in any conventional granulation equipment.

Objectionable tasting active pharmaceutical agents are not limited with the provision that it is used as a medically active component and has an objectionable taste. Examples of such drug include: central nervous system drugs such as a hypnotic sedative, a sleep inductor, an anxiolytic drug, an antiepileptic drug, an antipyretic-analgesics, an anti-inflammatory drug, an antidepressant, a histamine H2-antagonist, a 5 HT agonist, an antiparkinsonism drug, a psychoneurosis drug, digestive organ drugs such as an antidiarrheal drug, a drug for controlling intestinal infection, an antiulcer drug, a stomatic digestive drug and an antacid agent and allergy drugs such as an antitussive expectorant and a brochodilator and an anthelmintic, chemotherapy drug and drugs related to lipid metabolism disorders. The objectionable tasting drug is preferably selected from central nervous system drugs and digestive organ drugs.

Preferred objectionable tasting active pharmaceutical agents include, but are not limited to, loperamide, risperidone, ondansetron, granisetron, sumatriptan, naratriptan, olanzapine, lamotrigine, loratidine, fexofenadine, montelukast and desloratadine.

The objectionable tasting active pharmaceutical agents are present in the pharmaceutical composition in an amount of from about 0.1 to about 50 percent by weight. Unless otherwise mentioned all weight percentages disclosed herein are based on the final weight of the composition. Preferably, the objectionable tasting drug is present in the pharmaceutical composition in an amount of from about 0.5 to about 20 percent by weight.

Magnesium aluminium silicate is the primary taste-masking agent for the composition of the present invention and is mainly used intragranularly but it may also be used extra granularly also. The quantity of magnesium aluminium silicate is used in such an amount so as to be sufficient for taste masking without affecting the release of the drug from the dosage form.

Magnesium aluminium silicate is used in an amount of less than 20 percent by weight, preferably less than about 15 percent by weight. Extragranular magnesium aluminum silicate can optionally be used in combination with ion exchange resin and/or cyclodextrin to provide residual taste masking effect. Magnesium aluminum silicate is sold under the trade name Veegum®; by R. T. Vanderbilt Company, Inc. Magnesium aluminum silicate is also commercially available under the brand name Nusilin® from Fuji Chemicals, Japan. Magnesium aluminum silicate is also commercially available from Rennecker, USA under the brand name Magnabrite® F.

Polymers used for taste-masked composition of the present invention are essentially water insoluble. Examples of such polymers include but are not limited to ethyl cellulose, hydroxypropylmethyl cellulose phthalate, hydroxypropylmethyl cellulose acetate succinate, cellulose acetate phthalate, aminoalkyl methacrylate copolymer, hydroxypropylmethyl cellulose acetate succinate, methacrylic acid copolymer, cellulose acetate trimellitate (CAT), polyvinyl acetate phthalate and shellac. The preferable polymer is aminoalkyl methacrylate copolymer. Aminoalkyl methacrylate copolymer is exemplified by cationic co-polymer based on the dimethylaminoethyl methacrylate and neutral methacrylic acid esters, which are commercially available from Degussa Rohm Pharma Polymers under the brand names Eudragit® E100 and Eudragit® EPO (powdered form). Preferably, water insoluble polymer is used in an amount from about 2 to about 20% by weight.

The pharmaceutical composition of the present invention may further comprise diluents, binders, dispersing agents, disintegrants, surfactants, lubricants, glidants, flavoring agents, alkalizers, sweeteners and other pharmaceutical additives or excipient to facilitate the physical formulation of various dosage forms like orally disintegrating tablets, chewable tablets and suspensions (including dry powders or granules for suspension),

Diluents may be water-soluble or water insoluble. Examples of diluents include, but are not limited to, spray-dried or anhydrous lactose, sucrose, dextrose, starch, pre-gelatinized starch, mannitol, maltitol, sorbitol, xylitol, dextrin, microcrystalline cellulose, dibasic calcium phosphate, tribasic calcium phosphate and calcium sulphate. A preferred diluent is mannitol. A mixture of diluents may also be used. Mannitol is commercially available from under the brand name Pearlitol®. The diluent is preferably used in an amount of from about 10% to about 70% by weight.

The composition of invention may also comprise a surfactant. Examples of surfactant include, but are not limited to polyoxyethylene hardened castor oil, glyceryl monostearate, sorbitan monostearate, sorbitan monopalmitate, sorbitan monolaurate, polyoxyethylene polyoxypropylene block copolymers, polysorbates 80, sodium laurylsulfate, macrogols, sucrose esters of fatty acids. The preferred surfactant is sodium laurylsulfate. Preferably, surfactant is used in an amount of from about 0.1% to about 5% weight. A mixture of surfactants may also be used.

Examples of binder include, but are not limited to, methylcellulose, sodium carboxymethycellulose, calcium carboxymethycellulose, ethyl cellulose, hydroxypropyl methylcellulose, hydroxyethyl cellulose, hydroxypropyl cellulose, polyvinyl pyrrolidone, gelatine, polyvinyl alcohol, acacia, tragacanth, guar, pectin, starch paste, pre-gelatinized starch, and sodium alginate. A preferred binder is polyvinylpyrrolidone. A mixture of binders may also be used. The binder is preferably used in an amount of from about 2 to about 15% by weight.

Dispersing agents or dispersants help in dispersion of the powder resulting from the disintegration of orally disintegrating or dissolving tablet in the saliva present in the buccal cavity and help improve the mouth feel of the same. Suitable dispersing agents for the composition of the present invention are colloidal silicon dioxide, talc, magnesium stearate and titanium dioxide. The preferred dispersing agent is colloidal silicon dioxide. Preferably dispersing agent is used in an amount of from about 1 to about 5 percent by weight.

Examples of disintegrants include, but are not limited to, low substituted hydroxypropyl cellulose, carboxymethyl cellulose, calcium carboxymethyl cellulose, sodium carboxymethyl cellulose, starch, crystalline cellulose, hydroxypropyl starch, sodium alginate, partly pregelatinized starch, cross-linked sodium carboxymethylcellulose available as, e.g., Ac-di-sol®, Primellose®, Pharmacel® XL, Explocel® and Zymecels®ZSX, cross-linked polyvinylpyrrolidones, e.g., crospovidones, such as Polyplasdone® XL and Kollidon®CL. The combination of above-mentioned disintegrants can also be used. The preferred disintegrant is low substituted hydroxypropyl cellulose. The disintegrant is preferably used in an amount of from about 5% to about 50% by weight.

Examples of lubricants include, but are not limited to, vegetable oils, such as hydrogenated vegetable oil or hydrogenated castor oil; polyethylene glycols, such as polyethylene glycol (PEG)-4000 and PEG-6000; stearic acid; salts of stearic acid, such as magnesium stearate, sodium stearate and sodium stearyl fumarate; mineral salts, such as talc; inorganic salts; organic salts, such as sodium benzoate, sodium acetate and sodium oleate; and polyvinyl alcohols. The preferred lubricant is sodium stearyl fumarate. Preferably, lubricant is used in an amount from about 1 to about 5% by weight.

Examples of glidants include, but are not limited to, colloidal silica, magnesium trisilicate, amorphous silica, powdered cellulose, starch, talc and tribasic calcium phosphate. A preferred glidant is talc or colloidal silica or amorphous silica. For orally disintegrating or mouth dissolving dosage form of this invention colloidal silica performs the important function of cushioning agent thereby improving the mouth feel especially when formulation employs large particle size diluent like mannitol 200 S.D or mannitol 300 S.D. Glidants are used in extra concentrations in orally disintegrating tablets as they have additional function to perform i.e cushioning agent. Glidants are used in an amount from about 1 to about 30 percent by weight. Preferably, from about 5 to about 15 percent by weight.

It is within the scope of the invention to include various flavoring agent and sweeteners in the composition of the invention. Preferably, flavoring agents for the composition of the present invention are black current, sodium chloride, strawberry flavor, and peppermint flavor. Preferably, sweetners for the composition of the present invention include sucralose, acesulfame potassium and aspartame. A combination of sweeteners and flavoring agents can also be used. Preferably, flavoring agents are used in an amount of from about 0.5 to about 5 percent by weight. Preferably, sweeteners are used in an amount of from about 1 to about 5 percent by weight.

It is also with in the scope of the invention to include any alkalizers in the present composition to reduce the solubility of the drug in the saliva. Such alkalizers include sodium bicarbonate, sodium carbonate and magnesium carbonate. Preferred alkalizer is sodium bicarbonate. Preferably alkalizers are used in an amount of from about 1 to about 5 percent by weight.

The composition of invention is extremely palatable solid dosage form. The solid dosage form for the present invention includes sprinkles, capsules, caplets, powders and preferably monolayer tablets. Tablet may include multiple layer-compressed tablets, bilayer tablets, mouth dissolving tablets or orally disintegrating tablets, water dispersible tablets, and chewable tablets. More preferably tablets are orally disintegrating or dissolving tablets.

The tablets can be prepared by wet granulation which involve aqueous or non-aqueous solvents, dry granulation, and direct compression or by any other manufacturing technique known in the art. Preferably tablet is manufactured by wet granulation method. Compression of the lubricated blend can be carried out on a suitable tabletting machine having necessary tooling.

Wet granulation can be carried in conventional RMG mixer or planetary mixer. However more sophisticated equipment like fluid bed processor can be conveniently used for carrying out the granulation. For mixing of powders or granules, V-blender, octagonal blender, and double cone blender can be used. Drying of granules can be carried in fluid bed processor itself. Drying can also be carried out in conventional tray dryer.

The process for the said taste masked composition suitable for oral solid dosage form according to the present invention comprises of forming adsorbate of one or more active with an adsorbent such as magnesium aluminum silicate, mixing the resultant adsorbate with lubricants, dispersant, surfactant, sweeteners, disintegrants and sifting the mixture through suitable size mesh, granulating the resultant mixture with solution of water insoluble polymer, drying the resultant granules and sifting them through suitable size mesh, further blending the formed granules with fillers, lubricants, antiadherants, disintegrants, sweeteners, alkalizers and flavoring agents and compressing the blend into tablets of suitable shape or converting the resultant blend into suitable oral solid dosage forms.

The further process for preparing said taste masked composition suitable for oral solid dosage form according to present invention comprises of forming adsorbate of one or more active with an adsorbent such as magnesium aluminum silicate, mixing the resultant adsorbate with dispersant, lubricants, sweeteners, disintegrants and passing the mixture through suitable size mesh, granulating the resultant mixture with aqueous solution of binder and surfactant, followed by further granulation with solution of water insoluble polymer, drying the resultant granules and sifting them through suitable size mesh, further blending the formed granules with fillers, lubricants, antiadherants, disintegrants, sweeteners, alkalizers and flavoring agents and compressing the blend into tablets of suitable shape or converting the resultant blend into suitable oral solid dosage forms.

The following examples further illustrate the present invention. They are, however, not intended to be limiting the scope of the present invention in any way.

### Example 1

Taste masked pharmaceutical composition of Loperamide hydrochloride as presented in Table 1.

TABLE 1

| S. No | Ingredients | Qty (mg/tab) | Qty (%) |
|---|---|---|---|
| | Intragranular | | |
| 1 | Loperamide hydrochloride | 2 | 1.65 |
| 2 | Magnesium aluminium silicate | 14 | 11.60 |
| 3 | Colloidal silicon dioxide (Aerosil 200) | 5.0 | 4.15 |
| 4 | Aspartame | 3.0 | 2.49 |
| 5 | Sodium lauryl sulphate | 1.0 | 0.83 |
| 6 | L-Hydroxypropylcellulose (LH-11) | 20.0 | 16.60 |
| 7 | Polyvinylpyrrolidone (K-30) | 3.0 | 2.50 |
| 8 | Purified Water* | q.s | 0.0 |
| 9 | Eudragit EPO | 20.0 | 16.60 |
| 10 | Talc | 1.0 | 0.83 |
| 11 | Acesulfame potassium | 4.0 | 3.33 |
| 12 | Isopropyl alcohol* | q.s | q.s |
| 13 | Acetone* | q.s | q.s |
| | Extragranular | | |
| 14 | Colloidal silicon dioxide (Aerosil 200) | 0.5 | 0.41 |
| 15 | Sodium stearyl fumarate | 0.5 | 0.41 |
| 16 | Sodium chloride | 3.0 | 2.49 |
| 17 | L-Hydroxypropylcellulose (LH-11) | 5.0 | 4.15 |
| 18 | Mannitol (Pearlitol 300 DC) | 36.4 | 30.20 |
| 19 | Peppermint flavor | 0.8 | 0.66 |
| 20 | Strawberry flavor | 1.2 | 0.99 |
| | Total | 120.4 | |

*Not present in the final composition

### Manufacturing Procedure:

### Intragranular

1) The sodium lauryl sulphate and polyvinylpyrrolidone (K-30) were dissolved in sufficient quantity of purified water.

2) Loperamide hydrochloride, magnesium aluminium silicate, colloidal silicon dioxide, aspartame and L-HPC were blended together and sifted through suitable size mesh.

3) The blend obtained in stage (2) was granulated with the aqueous solution obtained in step (1) in RMG.

4) The granules were dried in fluid bed dryer.

5) Eudragit EPO was dissolved in 60:40 mixtures of isopropyl alcohol and acetone and talc is dispersed in this solution.

6) Acesulfame potassium was dissolved in sufficient quantity of purified water.

7) The granules of step 4) were coated with dispersion of stage 5) followed by solution of step 6) in fluid bed processor.

### Extragranular

8) Pearlitol 300 DC was sifted through suitable size mesh.

9) Colloidal silica, sodium stearyl fumarate, sodium chloride, L-HPC, strawberry flavor and peppermint flavor were sifted through suitable size mesh.

10) Ingredients of step 9) were blended with ingredient of step 8) and granules of step 7). And the blend so formed was compressed to give taste-masked tablet of loperamide hydrochloride.

### Example 2

Risperidone taste masked composition as presented in Table 2.

| S. No. | Ingredients | Qty (mg/tab) | Qty (%) |
|---|---|---|---|
| | Intragranular | | |
| 1 | Risperidone | 1.0 | 1.0 |
| 2 | Magnesium aluminium silicate | 6.0 | 6.0 |
| 3 | Colloidal silicon dioxide (Aerosil 200) | 3.0 | 3.0 |
| 4 | Aspartame | 4.0 | 4.0 |
| 5 | Sodium lauryl sulphate | 0.5 | 0.5 |
| 6 | Sodium stearyl fumarate | 1.0 | 1.0 |
| 7 | L-Hydroxypropylcellulose (LH-11) | 13.0 | 13.0 |
| 8 | Purified Water* | Q.s | q.s |
| 9 | Eudragit EPO | 8.0 | 8.0 |
| 10 | Talc | 1.0 | 1.0 |
| 11 | Acesulfame potassium | 2.0 | 2.0 |
| 12 | Isopropyl alcohol* | q.s | q.s |
| 13 | Acetone* | q.s | q.s |
| | Extragranular | | |
| 14 | Colloidal silicon dioxide (Aerosil 200) | 0.5 | 0.5 |
| 15 | Sodium stearyl fumarate | 1.5 | 1.5 |
| 16 | Sodium chloride | 2.0 | 2.0 |
| 17 | L-Hydroxypropylcellulose (LH-11) | 5.0 | 5.0 |
| 18 | Mannitol (Pearlitol SD200) | 49.5 | 49.5 |
| 19 | Peppermint flavor | 0.80 | 0.80 |
| 20 | Strawberry flavor | 1.20 | 1.20 |
| | Total | 100.0 | |

### Manufacturing Procedure:

### Intragranular

1) Sodium lauryl sulphate was dissolved in purified water.

2) Eudragit EPO and talc were dispersed in 60:40 mixtures of isopropyl alcohol and acetone.

3) Acesulfame potassium was dissolved in purified water.

4) Risperidone, magnesium aluminium silicate, colloidal silicon dioxide, L-HPC, aspartame and sodium stearyl fumarate were blended together and sifted through suitable size mesh.

5) Blend obtained in step 4) was first wetted with solution of step 1) and then first granulated with dispersion of step 2) followed by further granulation with solution of step 3) and subsequently the granules are dried in the fluid bed processor.

6) The dried granules were sifted through suitable size mesh.

### Extragranular

7) Colloidal silica, sodium stearyl fumarate, Sodium chloride, L-HPC, Strawberry flavor and Peppermint flavor were sifted through suitable size mesh.

8) Pearlitol SD 200 was sifted through suitable size mesh.

9) Ingredients of step 6) were blended with ingredient of step 7) & step 8) and the blend so formed was compressed into tablets or converted into other suitable orals solid dosage forms like dry powders, and granules for suspensions.

### Example 3

Taste masked composition of loperamide hydrochloride in Table 3:

| S. No | Ingredients | Qty (mg/tab) | Qty (%) |
|---|---|---|---|
| | Intragranular | | |
| 1 | Loperamide hydrochloride | 2.0 | 1.11 |
| 2 | Magnesium aluminium silicate | 14.0 | 7.78 |
| 3 | Colloidal silicon dioxide | 5.0 | 2.78 |
| 4 | Aspartame | 3.0 | 1.67 |
| 5 | Sodium lauryl sulphate | 5.0 | 2.77 |
| 6 | Sodium stearyl fumarate | 2.0 | 1.10 |
| 7 | L-Hydroxypropylcellulose (LH-11) | 18.0 | 10.0 |
| 8 | Polyvinylpyrrolidone (K-30) | 5.0 | 2.78 |
| 9 | Purified Water* | q.s | — |
| 10 | Eudragit ® EPO | 20.0 | 11.11 |
| 11 | Talc | 5.0 | 2.78 |
| 12 | Acesulfame potassium | 4.0 | 2.22 |
| 13 | Dibutylsebacate | 2.0 | 1.11 |
| 14 | Isopropyl alcohol* | q.s | — |
| 15 | Acetone* | q.s | — |
| | Extragranular | | |
| 16 | Colloidal silica (Aerosil 200) | 1.0 | 0.55 |
| 17 | Sodium stearyl fumarate | 1.0 | 0.55 |
| 18 | Sodium chloride | 3.0 | 1.66 |
| 19 | Beta Cyclodextrin | 8.0 | 4.44 |
| 20 | L-Hydroxypropylcellulose (LH-11) | 20.0 | 11.11 |
| 21 | Sodium bicarbonate | 5.0 | 2.78 |
| 22 | Granular Mannitol (Pearlitol160C)** | 55.0 | 30.56 |
| 23 | Peppermint flavor | 0.80 | 0.44 |
| 24 | Strawberry flavor | 1.20 | 0.67 |
| | Total | 180 | |

*Evaporates during processing.
**Pearlitol 160 C was granulated with aqueous solution of polyvinylpyrrolidone and granules formed were properly dried in fluid bed processor.

### Manufacturing Procedure:

### Intragranular

1) Sodium lauryl sulphate and polyvinylpyrrolidone (K-30) were dissolved in suitable quantity of purified water.

2) Loperamide hydrochloride, magnesium aluminium silicate, sodium stearyl fumarate, colloidal silicon dioxide, aspartame and L-HPC were blended together and sifted through suitable size mesh.

3) The blend obtained in stage (2) was granulated with the aqueous solution obtained in step (1) in RMG.

4) The granules were dried in fluid bed dryer and sifted through suitable size mesh.

5) Eudragit EPO was dissolved in 60:40 mixtures of isopropyl alcohol and acetone and talc and dibutyl sebacate is dispersed in this solution.

6) Acesulfame potassium was dissolved in sufficient quantity of purified water.

7) The granules of step 4) were coated first with dispersion of stage 5) followed by solution of step 6) in fluid bed processor.

### Extragranular

8) Pearlitol 160 C granules were sifted through suitable size mesh.

9) Colloidal silicon dioxide, sodium stearyl fumarate, sodium chloride, L-HPC, strawberry flavor and peppermint flavor, beta-cyclodextrin and sodium bicarbonate were sifted through suitable size mesh.

10) Ingredients of step 8) were blended with ingredient of step 9) and granules of step 7). The blend so formed was compressed into tablets or converted into other suitable oral solid dosage forms.

### Example 4

Taste masked pharmaceutical composition of Loperamide in Table 4:

| S. No | Ingredients | Qty (mg/tab) | Qty (%) |
|---|---|---|---|
| | Intragranular | | |
| 1 | Loperamide hydrochloride | 2.0 | 1.67 |
| 2 | Magnesium aluminium silicate | 10.0 | 8.34 |
| | (Magnabrite ® F) | | |
| 3 | Colloidal silicon dioxide | 3.0 | 2.5 |
| 4 | Aspartame | 2.5 | 2.1 |
| 5 | Sodium lauryl sulphate | 0.9 | 0.75 |
| 6 | Sodium stearyl fumarate | 2.0 | 1.67 |
| 7 | Crosslinked polyvinylpyrrolidone | 5.0 | 4.16 |
| 8 | Peppermint flavor | 0.8 | 0.67 |
| 9 | Purified Water* | q.s | — |
| | Extragranular | | |
| 10 | Colloidal silica (Aerosil 200) | 1.0 | 0.83 |
| 11 | Sodium stearyl fumarate | 1.0 | 0.83 |
| 12 | Crosslinked polyvinylpyrrolidone | 10.0 | 8.33 |
| 13 | Sodium bicarbonate | 5.0 | 4.17 |
| 14 | Mannitol (Pearlitol S.D. 200) | 75.4 | 62.83 |
| 15 | Black current | 0.4 | 0.33 |
| 16 | Aspartame | 1.0 | 0.83 |
| | Total | 120 | |

*Evaporates during processing

### Manufacturing Procedure:

### Intragranular

1) Sodium lauryl sulphate was dissolved in purified water.

2) Loperamide hydrochloride, Magnabrite® F, colloidal silicon dioxide, aspartame, sodium stearyl fumarate, crosslinked polyvinylpyrrolidone and peppermint flavor are blended together and sifted through suitable size mesh.

3) The blend obtained in step 2) was granulated with aqueous solution of step 1).

4) The granules were dried in tray dryer and sifted through suitable size mesh.

### Extragranular

5) Colloidal silica, sodium stearyl fumarate, crosslinked polyvinylpyrrolidone, sodium bicarbonate, black current, Pearlitol S.D. 200 and aspartame were sifted through suitable size mesh and mixed geometrically with granules of step 4)

6) Blend obtained in step 5) was compressed into tablets or converted into other oral solid dosage forms.

### Example 5

### Dissolution Studies

In order to assess the release of the drug (risperidone) from the dosage form (tablet) of example 2, the tablets were subjected to dissolution study using USP type II apparatus at 50 RPM. 500 ml of 0.1 N hydrochloric acid was used as dissolution media. The results of study were presented in table 5.

TABLE 5

| | Percentage of drug (risperidone dissolved) in: | | | | |
|---|---|---|---|---|---|
| Tablet No. | 5 min | 10 min | 15 min | 20 min | 30 min |
| 1 | 96 | 98 | 100 | 100 | 99 |
| 2 | 96 | 96 | 97 | 97 | 97 |
| 3 | 95 | 96 | 96 | 98 | 96 |
| 4 | 100 | 101 | 101 | 101 | 101 |
| 5 | 101 | 104 | 102 | 102 | 102 |
| 6 | 100 | 100 | 101 | 100 | 100 |
| Mean | 98 | 99 | 100 | 100 | 99 |

### Example 6

### Evaluation of Taste Masking Effect

The taste-masked tablet of example 2 was evaluated for taste masking of the objectionable tasting risperidone. The tablet of the invention was tasted by to six healthy volunteers. The taste-masked tablet of risperidone was put on tongue of each volunteer separately. The response from each volunteer was recorded and quantified on a scale of 10. Grading of response was quantified as follows.

| | |
|---|---|
| Very objectionable (Immediate spitting) --- | 0 |
| Objectionable taste --- | 1 |
| Moderate taste- | 5 |
| Good taste - | 7 |
| Very good taste --- | 9 |
| Excellent taste | 10 |

Responses from the volunteers were recorded as follows:

TABLE 6

| Volunteer | Response | Quantified Response |
|---|---|---|
| First | Good palatability | 7 |
| Second | Moderate taste | 5 |
| Third | Good palatability | 7 |
| Fourth | Good palatability | 7 |
| Fifth | Very good palatability | 9 |
| Sixth | Good palatability | 7 |
| Mean | Good palatability | 7 |

From the results of the example 5 and example 6 it is evident that composition of invention has good tasting effect and at the same time release of the drug from the dosage form is not hindered.

While this invention has been described in detail with reference to certain preferred embodiments, it should be appreciated that the present invention is not limited to those precise embodiments rather, in view of the present disclosure, which describes the current best mode for practicing the invention, many modifications and variations, would present themselves to those skilled in the art without departing from the scope and spirit of this invention.

1-35. (canceled)

36. A solid dosage form in the form of a tablet for oral administration comprising:
(a) from about 0.5 to 50% by weight of risperidone or its pharmaceutically acceptable salts, solvates or hydrates,
(b) less than about 15% by weight of magnesium aluminum silicate, and
(c) from about 2 to 20% by weight of aminoalkyl methacrylate copolymer, and
(d) optionally other pharmaceutically acceptable excipients, and
wherein all the above ingredients are granulated prior to compression into tablets.

37. A solid dosage form in the form of a tablet for oral administration comprising:
(a) from about 0.5 to 50% by weight of 5-HT agonist or its pharmaceutically acceptable salts, solvates or hydrates,
(b) less than about 15% by weight of magnesium aluminum silicate, and
(c) from about 2 to 20% by weight of aminoalkyl methacrylate copolymer, and
(d) optionally other pharmaceutically acceptable excipients, and
wherein all the above ingredients are granulated prior to compression into tablets.

38. A solid dosage form of claim 36, wherein aminoalkyl methacrylate copolymer is cationic copolymer of dimethylaminoethyl methacrylate and neutral methacrylic acid esters.

39. A solid dosage form of claim 36, wherein pharmaceutically acceptable excipients are selected from diluents, binders, disintegrants, surfactants, lubricants, glidants, flavoring agents, alkalizers and sweeteners or combination thereof.

40. A solid dosage form of claim 37, wherein the oral solid dosage form is an instant release tablet.

41. A solid dosage form of claim 40, wherein the oral solid dosage form is an orally disintegrating tablet.

42. A solid dosage form of claim 40, wherein the oral solid dosage form is a chewable tablet.

43. A solid dosage form of claim 40, wherein the oral solid dosage form is a monolayer or bilayer or multiple layer tablet.

44. A process for preparing solid dosage form of claim 36, the process comprising:
(a) blending risperidone with magnesium aluminum silicate, colloidal silicon dioxide, L-HPC, aspartame and sodium stearyl fumarate and sifting the blend so formed,
(b) granulating the blend of step (a) with aqueous solution of sodium lauryl sulphate, followed by
(c) granulating with dispersion of aminomethacrylate copolymer and talc in mixture of isopropyl alcohol and acetone, and subsequently
(d) granulating with aqueous solution of acesulfame,
(e) blending the granules of step (d) with sifted colloidal silica, sodium stearyl fumurate sulphate, sodium chloride, L-HPC, strawberry flavor, peppermint flavor and mannitol, and
(f) compressing the blend into tablets.

45. A process for preparing solid dosage form of claim 37, the process comprising:
(a) blending 5HT agonist drug with magnesium aluminum silicate, colloidal silicon dioxide, L-HPC, aspartame and sodium stearyl fumarate and sifting the blend so formed,
(b) granulating the blend of step (a) with aqueous solution of sodium lauryl sulphate, followed by
(c) granulating with dispersion of aminomethacrylate copolymer and talc in mixture of isopropyl alcohol and acetone, and subsequently
(d) granulating with aqueous solution of acesulfame,
(e) blending the granules of step (d) with sifted colloidal silica, sodium stearyl fumurate sulphate, sodium chloride, L-HPC, strawberry flavor, peppermint flavor and mannitol, and
(f) compressing the blend into tablets.
